(19) United States
(12) Patent Application Publication
(10) Pub. No.: US 2015/0313477 A1
(43) Pub. Date: Nov. 5, 2015

(54) MEDICAL DEVICE SYSTEM

(71) Applicant: Albert Maarek, Miami, FL (US)

(72) Inventor: Albert Maarek, Miami, FL (US)

(21) Appl. No.: 14/681,297

(22) Filed: Apr. 8, 2015

Related U.S. Application Data
(63) Continuation-in-part of application No. 14/259,282, filed on Apr. 23, 2014, now abandoned.
Application No. 14/259,282 is a continuation of application No. 13/514,353, filed on Oct. 10, 2012, now abandoned.
(60) Provisional application No. 61/267,510, filed on Dec. 8, 2009.
(60) Provisional application No. 61/267,542, filed on Dec. 8, 2009.

(51) Int. Cl. A61B 5/0205; A61B 5/0295; A61B 5/00; A61B 5/1455

(52) U.S. Cl. CPC A61B 5/0205; A61B 5/14552; A61B 5/0295; A61B 5/7278; A61B 5/7282; A61B 5/4848; A61B 5/02225

(57) ABSTRACT

The invention relates to a medical device system comprising at least two technologies wherein at least one technology is based on galvanic skin response measurement and at least one technology is based on oximeter wave form measurement wherein software cross analyzes the results to assess a level of homeostasis and presents a homeostasis score. Preferably a pulse oximeter provides a vascular waveform using photoelectrical plethysmography in spectral analysis. Galvanic skin response is assessed using a change in voltage polarity during measurement. Preferably the system includes a) galvanic skin response, b) bioimpedance in tetrapolar mode c) oximeter sensor and d) blood pressure using oscillometric measurements.

This application is a Continuation in Part of U.S. Ser. No. 14/259,282 and also claims the benefit of PCT applications IB2010/003114, IB2013/002595 and IB2014/001047.

The present invention relates to a medical device system utilizing a combination of technologies and software to establish a health assessment. More particularly the device comprises technologies including oximeter, galvanic skin response, impedance measurement and blood pressure measurements and software using signal processing analysis and calculation of score to establish a measure of human homeostasis (human body regulatory mechanisms).

Homeostasis is supported by 2 main functions: autonomic nervous and endothelial functions.

The autonomic nervous system (ANS) is an extensive neural network whose main role is to regulate the internal environment and body functions by controlling homeostasis which includes hemodynamics, blood pressure, heart rate, blood glucose level, sweating and visceral functions. The ANS acts through a balance of stimulation or inhibition of its own two components—the sympathetic and parasympathetic nervous systems. Sympathetic and parasympathetic branches act via neurotransmitters and receptors activation.

The endothelial function is related to the ability of the blood vessels to dilate when necessary. Endothelial dysfunction can be defined as reduced bio-availability of Nitric Oxide (NO), which plays many roles in maintaining vascular health, most importantly its role in vasomotor. Hence, endothelial dysfunction is defined as an impairment of endothelium dependent vasodilation.

As stated by Lippincott (Medical encyclopedia): “Disease or death is often the result of dysfunction of internal environment and regulatory mechanisms. Understanding the body's processes, responses and functions is clearly fundamental to the intelligent practice of medicine.”

At present, the clinical context, the lab tests, functional tests such as EKG or Doppler and imagery provide doctors data to establish diagnoses and treatment plans on predictions based upon recognized scientific background and practitioner decision.

However, these analyses do not take into account the overall potential of regulation any individual patient. An overall homeostasis evaluation which represents a patient's potential adaptation to a dysfunction or disease should enhance a treatment plan.

It is an aim of the present invention to provide a combination of devices comprising different technologies and dedicated software to establish an overall potential of auto regulation of the patient. It is a further aim to assign a score to be known as the homeostasis score.

The homeostasis score provides a fast overview of a patient's homeostasis processes and responses with the key indicators, to understand the patient's potential adaptation to lifestyle, disorders, diseases and any current treatments.

The proposed technology and its analysis aim to provide effective cost therapeutic adjustment and follow up. With the adjunct of the homeostasis evaluation, a doctor should be able to test how the planned treatment would affect a patient, save time and as the possibilities of treating diseases improve, it is important to choose the right treatment for each individual patient.

According to the present invention there is provided a device wherein at least two technologies based on galvanic skin response and oximeter measurements and software signal processing analysis and cross analyses the results to assess the homeostasis score of an individual.

In one particular preferred embodiment the system is a combination of 4 technologies with 6 features and signal processing analysis managed by software.

Preferred technologies include a) Galvanic skin response, b) bio impedance in tetra polar mode, c) oximeter wave form and d) blood pressure oscillometric measurements.

Preferred bio impedance biosensor features:
1. The Galvanic Skin response feature evaluates the segmental and general conductance of the human body with direct current via at least 2 to 6 tactile electrodes. The signal processing analysis of the measurement provides sudomotor function assessment which is related to the function of the sympathetic cholinergic division of the ANS.
2. The bio impedance in tetra polar mode feature evaluates the resistance and the reactance of the human body using a mono frequency (50 KHz) via 4 tactile electrodes, to estimate body composition parameters (total body water (TWB), fat free mass, fat mass) according to predictive equations as commonly seen in peer reviews.
3. Oximeter wave forms or photoelectrical plethysmography (PTG): PTG uses transmitted infrared and red light to measure relative blood volume in the fingertip. PTG waveforms are reflective of blood movement in cutaneous vessels and can be used to identify synchronous depolarization of cardiovascular tissue. The fundamental frequency of the PTG waveform, typically around 1 Hz reflects the heart rate. Lower frequency components such as respiratory, thermoregulatory and sympathetic nervous system effects are also contained within the PTG signal.
4. Blood pressure measurements. The non-invasive blood pressure device feature is using oscillometric method and measures the systolic and diastolic pressure at the arm only or simultaneous at arms and ankles.

The invention will now be described with reference to the accompany non-limiting figures wherein

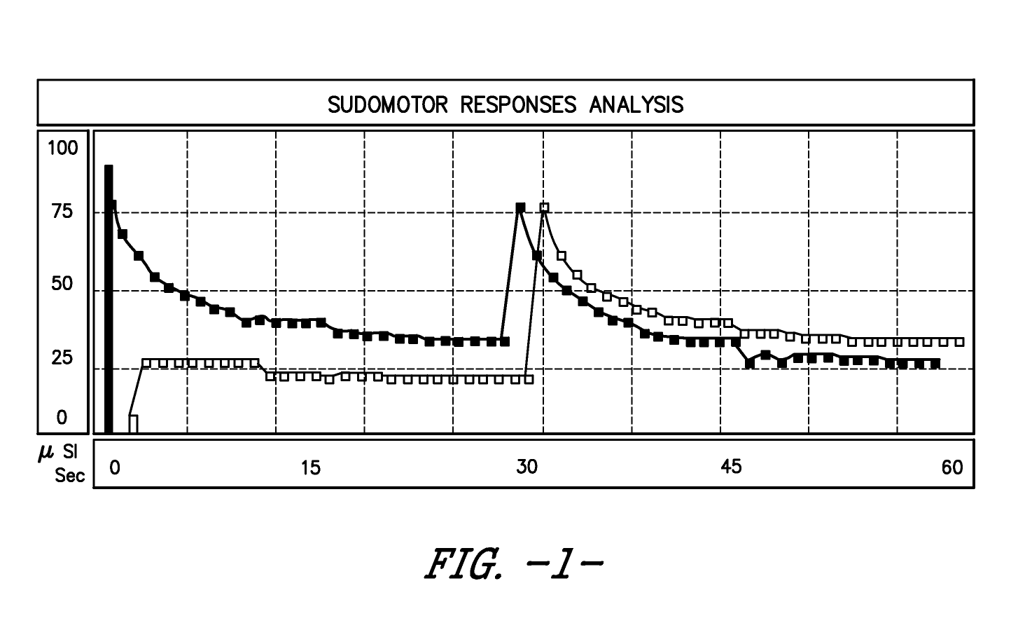

FIG. 1 shows the galvanic skin response records

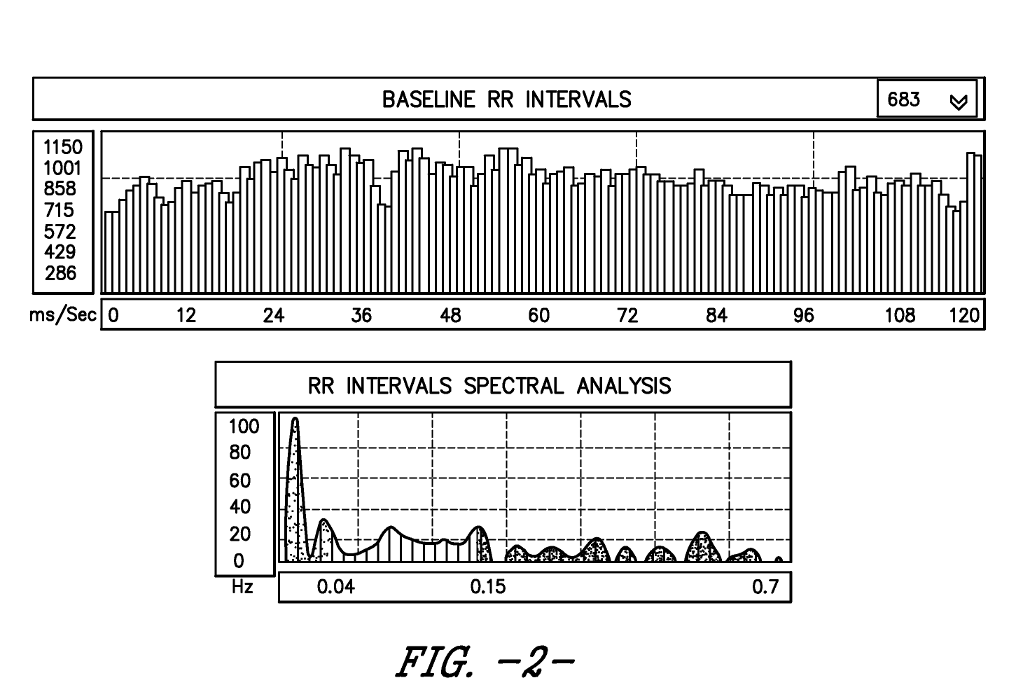

FIG. 2 shows the records of HRV signal in time domain and frequency domain analysis

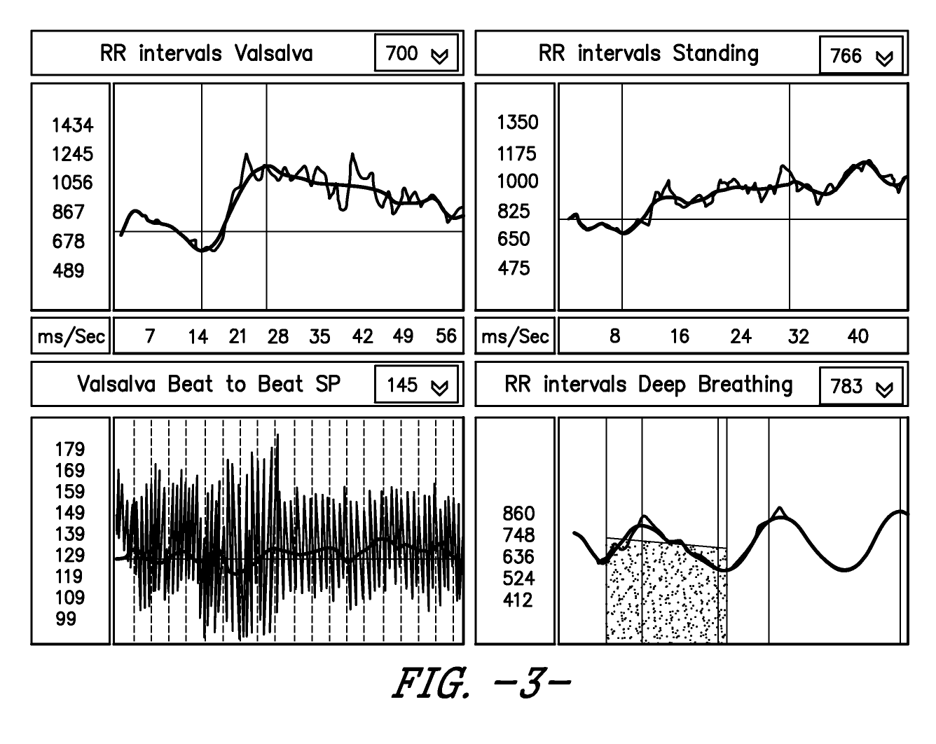

FIG. 3 shows the records of HRV and change in blood pressure during some Ewing tests (Valsalva maneuver, deep breathing and change in posture).

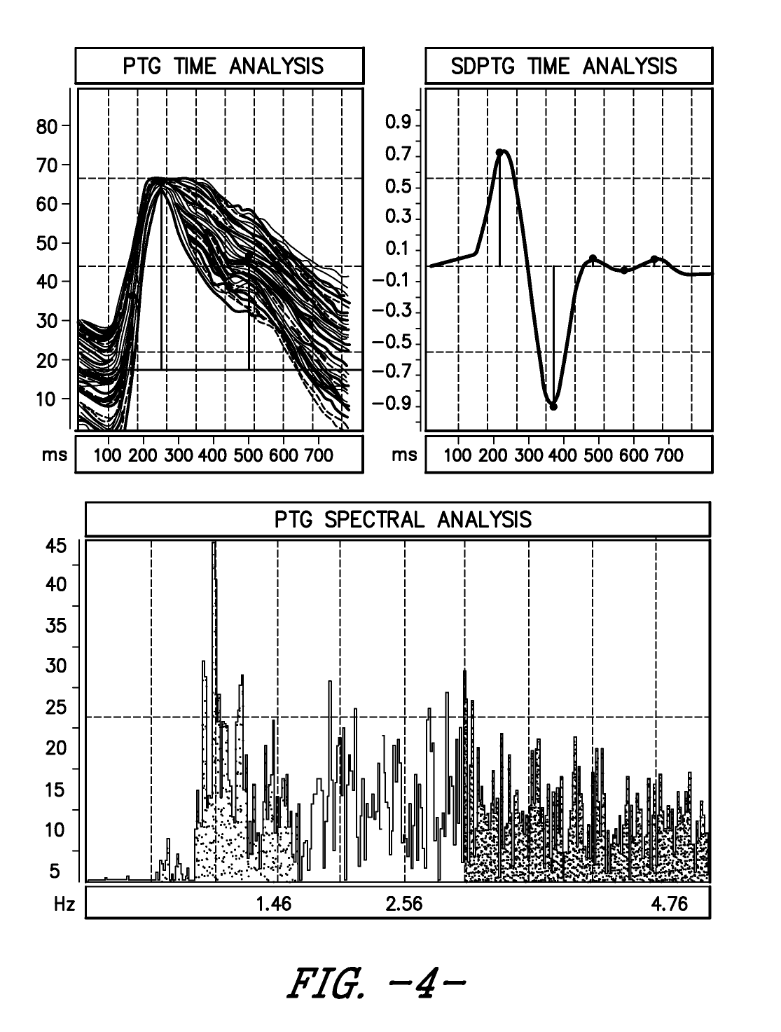

FIG. 4 shows the records of PTG analysis in time analysis and frequency domain

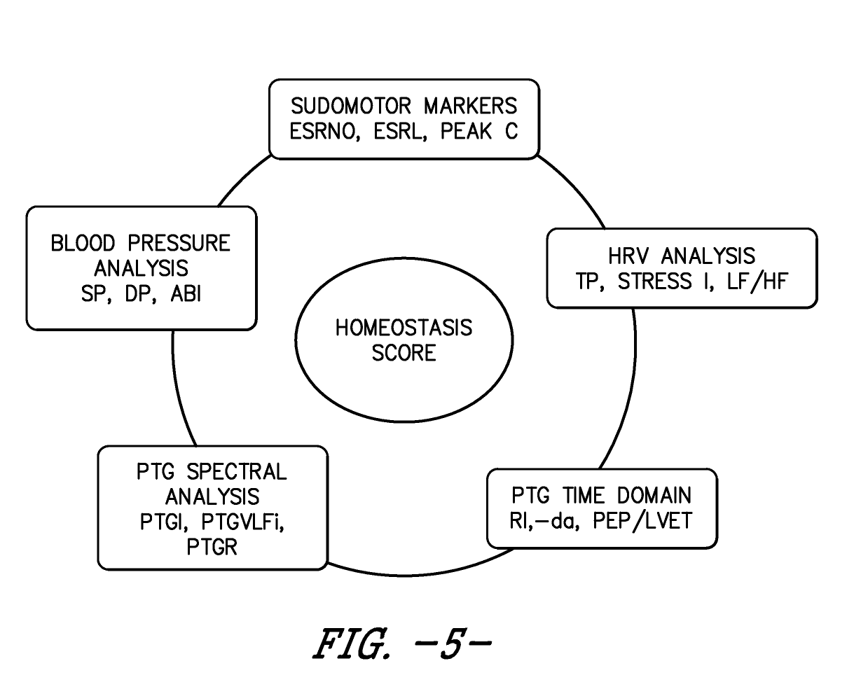

FIG. 5 shows the various markers contributing to the calculation of the homeostasis score

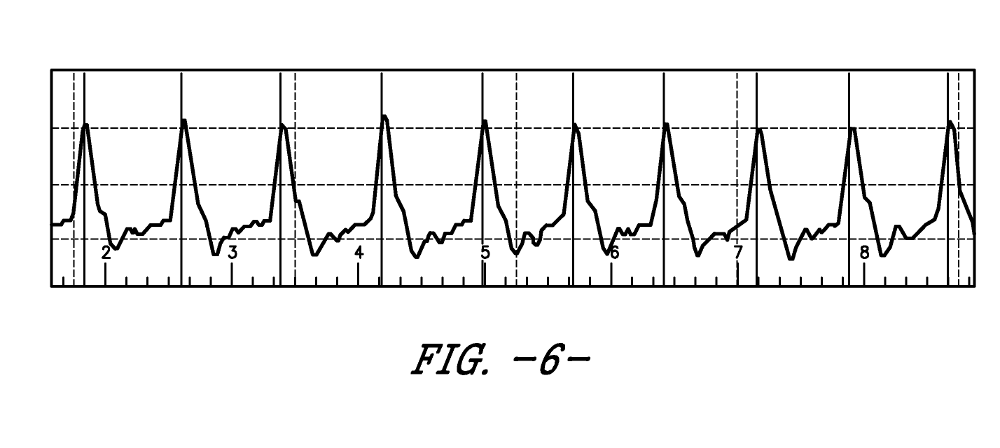

FIG. 6 shows the first derivative of a PTG waveform. Instantaneous heart rate can be derived by calculating the time between two peaks of the first derivative.

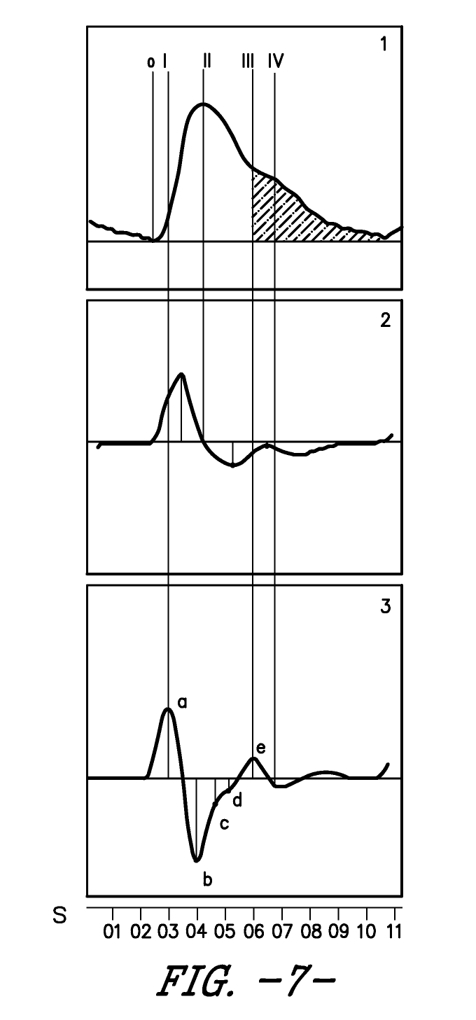

FIG. 7 shows An averaged PPG wave (1) and its first (2) and second (3) derivatives.

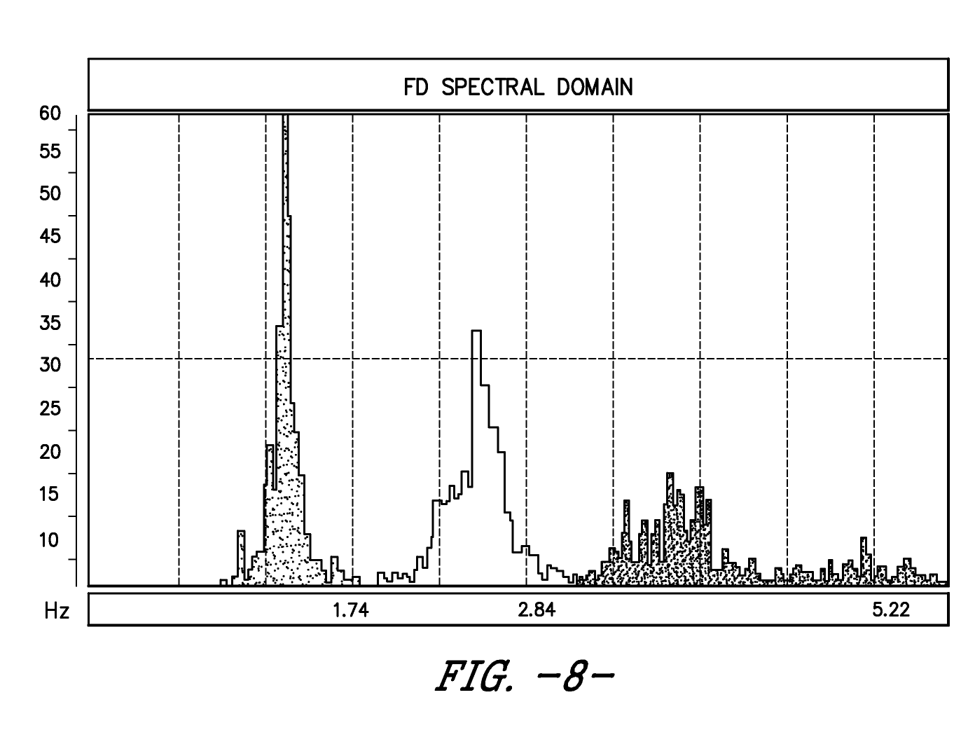

FIG. 8 shows Homeostatic markers: Fast-Fourier-Transform of a photoplethysmograph waveform. The waveform in the frequency domain is separated into three frequency bands: the ‘very low frequency’, the ‘low frequency’, and the ‘high frequency’.

### GALVANIC SKIN RESPONSE (GSR) SIGNAL PROCESSING ANALYSIS

Measurements are typically made with weak Direct Current between 2 tactile electrodes placed symmetrically on skin with the higher density of sweat glands of the subject (i.e. palm of hands, sole of feet or forehead)

The hand and foot electrodes are typically at least 250 cm2 and in stainless steel

The forehead electrodes are typically disposable (single use) and preferably in AgAgCl.

Each electrode is alternatively cathode then anode (change in voltage polarity), which permits in the particular embodiment described the recording of the intensity/voltage/resistance and conductance (Law of Ohm) of each pathway (volume of the body between 2 electrodes) of the human body.

The galvanic skin response generates positive (over the ground) and negative (below ground) voltage.

From left to right electrodes, the voltage is negative and from right to left electrodes, the voltage is positive.

The galvanic skin response measurement process is an improved sympathetic skin response method following a constant electrical stimulation, and change the direction of the voltage at the middle of the measurement with the following sequence:

Right to left foot (+1.28V) 15 seconds/change in current direction from left to right foot (−1.28V) 15 seconds and measurement of the right foot electrode.

Right to left hand (+1.28V) 15 seconds/change in current direction from left to right hand (−1.28V) 15 seconds and measurement of the right hand electrode.

Left to right foot (−1.28V) 15 seconds/change in current direction from right to left foot (+1.28V) 15 seconds and measurement of the left foot electrode.

Left to right hand (−1.28V) 15 seconds/change in current direction from right to left hand 15 (+1.28V) seconds and measurement of the left hand electrode.

The change in direction of the voltage provides a difference of potential (voltage) of peak of voltage (negative+positive output voltage on the bulk of the electrodes)

GSR provides a quantitative evaluation of the sweat response according to the electrochemical oxidation measurements on the bulk of the left electrodes and according to the carried energy in electric fields on the right electrodes (when the negative voltage is applied, the electrical measurement is not generated by electrons or ions).

Proposed Conductance and Electrolysis Analysis

Since sweat is a dilute sodium chloride (NaCl) solution, and a constant weak direct current of 1.28 V is sent between 2 electrodes, the general principle of Electrolysis of Aqueous NaCl is applicable. Sweat comprises 99.2 to 99.6% of water and 0.2 to 0.5% of NaCl.

The electrochemical window is defined by both reduction and oxidation according to the following reactions:

At the right electrodes: The output voltage is negative

The electrical measurement is not generated by ions.

Therefore we get only a release of water (H2O has a reduction potential of −0.83 V).

The measured electrochemical reduction half reaction related to the sweat production occurring at the cathode is:

2H2O+(2e−)→H2(g)+(2OH—) when the output voltage at the passive responding electrode is <=−0.83V which is corresponding to 65 μSi.

And produces hydrogen gas and OH— ions

The measured conductance (average of maximum conductance and conductance at 15 second after the maximum) at the right electrodes is named ESR NO (Electro Skin response-Nitric Oxide) because no ions are released and therefore the reaction is only related to the vasodilation response of surrounding vessel induced by the electrical stimulation we suggest the more probable assumption that ESRNO could be a valid marker of the microcirculatory state and it is our first marker of the sudomotor function. ESRNO <65 μSi is considered as lower sweat response and could be a sign microcirculatory disorder.

At the left electrodes: The output voltage is positive

Cl— ions could not be oxidized at the positive electrode for 2 reasons:
a. The voltage applied is not thermodynamically sufficient to drive electrolysis (Cl— ions have an oxidation potential of 1.39V and the generator produces 1.28V)
b. There is a competition between the negative ions at the positive anode. The Cl— ions compete with the hydroxide (OH—) ions to release their electrons to the anode. Regarding the very low concentration of chloride ions in the sweat (0.2 to 0.5%), and very high concentration of OH— ions from the water electrolysis, only, OH— are released at the anode.
OH— has an oxidation potential of 0.40 V and H2O has an oxidation potential of 1.23 V.

The measured electrochemical oxidation half reactions occurring at the anode are:
4OH—→2H2O+O2(g)+4e− when the output voltage at the passive responding electrode is >=0.40V which is corresponding to 37 μSi
2H2O→O2(g)+4H++4 e− when the output voltage at the passive responding electrode is >=1.23V which is corresponding to 90 μSi

And produce water, oxygen gas and H+ ions.

We identified at the left electrode 2 markers:

The Peak Conductance at the Left Electrode is the Main Marker of the Sudomotor Function

The change in voltage direction increases the output voltage according to the patient skin resistance, and therefore provides a peak of conductance (Peak C).

The measured voltage of the responding passive left electrode <0.40 V (37 μSi) is a marker of absence of sweat response at the anode and significant sudomotor dysfunction.

The measured voltage of the responding passive electrode left electrode <1.23V (90 μSi) and >0.40 V (37 μSi) is a marker of a reduced sweat response at the anode and sudomotor dysfunction.

Latency

The measured time from the change of voltage polarity to the Peak C at the left electrode is named ESR L (Electro Skin response Latency) and it is our last marker of the sweat response at the anode and ESR L>2 seconds is the third marker of sudomotor dysfunction.

In the present invention, the 3 markers (ESRNO, ESRL and Peak C) will be considered in the Homeostasis score calculation.

Bio Impedance Analysis

This technology is well known as well as the body mass Index calculation.

The resistance and reactance calculate will be converting in estimated body composition parameters (TWB, Fat Free mass, fat mass) according to the predictive equations of BIA (Body Impedance Analysis) issue from the peer reviews.

In the prevent invention, BMI and Fat Mass (FM) will be considered in the Homeostasis score calculation.

Oximeter Signal Processing Analysis

One method for monitoring cardiovascular events and peripheral circulation is through photoplethysmographic (PTG) analysis. PTG uses transmitted infrared and red light to measure relative blood volume in the fingertip. PTG waveforms are reflective of blood movement in cutaneous vessels and can be used to identify synchronous depolarization of cardiovascular tissue. The fundamental frequency of the PTG waveform, typically around 1 Hz reflects the heart rate. Lower frequency components such as respiratory, thermoregulatory and sympathetic nervous system effects are also contained within the PTG signal [2]. Arterial stiffness, indicative of endothelial dysfunction, may also be measurable from calculations made using the PTG waveforms analysis [2]. These measurements do not require lengthy examinations and are non-invasive approaches to identify abnormal cardiovascular function, possibly stemming from ANS and endothelial dysfunction.

PTG Time Domain Analysis

Throughout the entirety of the PTG waveform, relative changes in a patient's heart rate can be extracted by calculating the peak-to-peak interval. In order to find the peak-to-peak interval with high accuracy, in this study we extracted local maximum of the first derivatives. In FIG. 6 we have the first derivative of a PTG waveform, with lines corresponding to the local maximum of the first derivative from several distinct cardiac cycles.

The time between these points is defined as the peak-to-peak interval, and is calculated based on the sampling frequency and the number of samples collected between the two defined peaks. The sampling frequency of the pulse oximeter used in this study is 60 Hz.

First and second derivatives of the PTG waveform can aid in understanding a single or average PTG wave. In FIG. 7 we have an averaged PTG wave (FIG. 7.1) created from several extracted waves in the full PTG waveform. By calculating the first derivative (FIG. 7.2) and second derivative (FIG. 7.3), we can display measurements of various cardiac events. The time between points I and the preceding trough (*) in 1 corresponds to an estimate of the pre-ejection period (PEP). Point I is extracted from the second derivative point a. Point III in 1 corresponds to the dichrotic notch, separating systole (yellow) and diastole (purple) phases of the cardiac cycle and is extracted from point e in the second derivative [5]. The time between point II and point I corresponds to estimates of the left-ventricular-ejection-time (LVET) [6]. Point IV in 1 corresponds to the diastolic peak, extracted from the trough following point (e) in the second derivative [6].

Additionally, ratios of the amplitude of (a) to amplitudes of b, c, d, and e, can aid in understanding arterial stiffness. Studies have shown that ratios |b/a| and |d/a| decrease in aging populations and some studies have linked these ratios directly to arterial distensibility [5,6]. The ratio c/a has been linked to hypertension and has also been found to decrease with age [7,8]. Ratio d/a specifically may be useful in evaluating vasoactive agents and left ventricular afterload [7]. Takazawa et al. used an index (b-c-d-e)/a to evaluate peripheral vascular aging and noted potential use for screening atherosclerosis [9]

PTG Spectral Analysis

The Fourier transform of the PTG recording could reveal information regarding autonomic activity and heart rate variability. In FIG. 8, a healthy patient's fast-Fourier transform of a two minute PTG recording shows the characteristic peaks and frequency bands of a healthy patient. From this information, average heart rate, power of each frequency band, peak amplitudes and their corresponding frequency bins, and total power can be extracted. In this study, we will discuss diagnostic markers based off of the Fourier transformed PTG signal. Harmonic components included in the PTG waveform and elucidated through spectral analysis include those associated with heart rate variability, respiratory effects on the cardiac cycle, systolic and diastolic effects on peripheral blood flow, and the ability for cutaneous blood vessels to dilate and contract. Clinical studies showed the correlation between PTG spectral analysis markers with endothelial dysfunction as well as with the autonomic nervous system (ANS) dysfunction or failure. Since, endothelial and ANS function control the body homeostasis, we called the PTG spectral analysis parameters as Homeostatic markers: PTG index (PTGi) is the sum of all amplitudes of the graphic, PTG very low frequency Index (PTGVLFi) is the ratio of the area PTG very low frequency (VLF)/ESRNO and PTG Ratio (PTGR) is the ratio of PTGVLF/PTGi.

In the present invention in order to calculate the homeostasis score we focus on the following markers:
a. Total Power and LF/HF calculation from the HRV in spectral analysis
b. Stress Index calculation from the HRV in time domain analysis.
c. Ratio PTG Reflexion Index (RI) from PTG analysis
d. negative |d/a| from PTG second derivative analysis
e. PEP/LVET from PTG and PTG second derivative analysis
f. PTGi from PTG Spectral Analysis
g. PTGVLFi from PTG Spectral analysis and galvanic skin response marker at the negative electrode
h. PTGR from spectral analysis.

Blood Pressure Oscillometric Measurements
This type of device is in routine and does not need more clinical data and validation when perform only on the arm.
When the measurements are performed in simultaneous in arms and ankles, the Ankle branchial Index (ABI) could be calculated.

In the prevent invention, Systolic and diastolic pressure measuring in arm and ABI will be considered in the Homeostasis score calculation.

Calculation of the Homeostasis Score.

Homeostasis score is using markers of the software signal analysis of the combination of devices described above, the markers are:

Sudomotor Score

Each sudomotor marker (ESRNO, ESRL and Peak C) is scored as follow: normal range=2, borderline=1 and abnormal=0.

Body Composition Score

Normal range=5

FM>N+BMI<=29=4

FM<N=3

FM>N+BMI>29 and <=35=2

FM>N+BMI>35=1

Heart Rate Variability (HRV) Score

Each HRV Marker (total Power, Stress Index and LF/HF) is scored as follow: normal range=2, borderline=1 and abnormal=0.

PTG Score:

Each PTG Marker (RI, -da, PEP/LVET, PTGi, PTGVLFi and PTGR) is scored as follow: normal range=2, borderline=1 and abnormal=0.

Blood Pressure Scores

Systolic and diastolic at the arm:

Systolic<=120 Diastolic<=80=4

Systolic<=121-139 Diastolic<=81-89=3

Systolic<=140-159 and/or Diastolic<=90-99=Class 2

Systolic<=>160 and/or Diastolic>100=1

ABI>1 and <1, 40=0

ABI<0.9 and >0.6=1

ABI<=0.6=2

ABI>=1.40=3

Therefore, lower homeostasis score, higher the homeostasis of an individual is degraded and potential of adaptation reduced.

Best Score=0

If we use all technologies: Worth Score=35

Clinical applications and studies using markers of homeostasis score

Study 1

A cross-sectional assessment to detect type 2 diabetes with endothelial and autonomic nervous system markers using a novel system

Background: Type 2 diabetes mellitus is frequently unrecognized until complications appear. Diabetic autonomic neuropathy is one of the early complications of type 2 diabetes mellitus, resulting in autonomic nervous system (ANS) dysfunction. The purpose of this study was to determine the validity of ANS function indicators to screen for type 2 diabetes mellitus, as measured by the TM-Oxi and SudoPath system.

Methods: All enrolled participants completed a basic sociodemographic and medical history questionnaire including current medications. Healthy controls (n=25) underwent a 2-hour oral glucose tolerance test (OGTT) to evaluate glucose, insulin, and insulin C-peptide. Patients with type 2 diabetes mellitus (n=24) were assessed with fasting plasma glucose (FPG) and glycosylated hemoglobin. The TM-Oxi and SudoPath system evaluation was completed by all subjects. Data were analyzed using SPSS 22. Frequency and descriptive statistics were calculated on all variables. The criterion for statistical significance was α=0.05.

Results: The twenty-five healthy controls had a mean age of 37.0 years. The twenty-four type 2 diabetes mellitus patients currently undergoing standard treatment had a mean age of 48.9 years. Based on the American Diabetes Association guidelines, we detected pre-diabetes in 4 subjects and diabetes in 1 subject, while all other subjects had normal FPG values. At 120 minutes, the correlations between the OGTT and cardiometabolic risk score (CMRS) were:

r=0.56 (p=0.004) for glucose and r=0.53 (p=0.006) for insulin. At 120 minutes, the correlations between the OGTT and photoplethysmography index (PTGi) were: r=−0.56 (p=0.003) for glucose and r=−0.41 (p=0.04) for insulin. The CMRS, PTGi, and plethysmography total power index (PTGVLFi) differed significantly between the diabetes patients and healthy participants. The specificity and sensitivity for the CMRS, PTGi, and PTVLFi comparing the diabetes patients with healthy controls were high.

Conclusion: The TM-Oxi and SudoPath system shows promise as a valid, convenient, and non-invasive screening method for type 2 diabetes mellitus. The ANS function and CMR indicators measured by this system may be useful in guiding diabetes and cardiovascular health screening, treatment, and monitoring.

Study 2

The spectral analysis of photoplethysmography to evaluate an independent cardiovascular risk factor.

Background:

In this study, we evaluate homeostatic markers correlated to autonomic nervous and endothelial functions in a population of coronary artery disease (CAD) patients versus a control group. Since CAD is the highest risk marker for sudden cardiac death, the study objective is to determine whether an independent cardiovascular risk score based on these markers can be used alongside known conventional cardiovascular risk markers to strengthen the understanding of a patient's vascular state.

Materials and Methods:

Sixty-five subjects (13 women) with a mean age of 62.9 years (range 40-80 years) who were diagnosed with CAD using coronary angiography (group 1) and seventy-two subjects (29 women) with a mean age of 45.1 years (range 18-85 years) who claimed they were healthy (group 2) were included in the study. These subjects underwent examination with the TM-Oxi and SudoPath systems at IPC Heart Care Centers in Mumbai, India. The TM-Oxi system takes measurements from a blood pressure device and a pulse oximeter. The SudoPath measures galvanic skin response to assess the sudomotor pathway function. Spectral analysis of the photoplethysmograph (PTG) waveform and electrochemical galvanic skin response allow the TM-Oxi and SudoPath systems to calculate several homeostatic markers, such as the PTG index (PTGi), PTG very low frequency index (PTGVLFi), and PTG ratio (PTGr). The focus of this study was to evaluate these markers (PTGi, PTGVLFi, and PTGr) in CAD patients against a control group, and to calculate an independent cardiovascular risk factor score: the PTG cardiovascular disease risk score (PTG CVD), calculated solely from these markers. We compared PTGi, PTGVLFi, PTGr, and PTG CVD scores between the CAD patient group and the healthy control group. Statistical analyses were performed using receiver operating characteristic curves to determine the specificity and sensitivity of the markers to detect CAD at optimal cutoff values for PTGi, PTGVLFi, PTGr, and PTG CVD. In addition, correlation analyses between these markers and conventional autonomic nervous system and endothelial function markers were performed to understand the possible underlying physiological sources of the differences observed in marker values between CAD patients and healthy control patients. Additionally, t-tests were performed between two subgroups of the CAD patient group to determine whether diabetic or coronary artery bypass grafting (CABG) patients have significantly different PTGi marker values.

Results:

Each spectral analysis PTG marker yielded a high specificity and sensitivity to detect CAD. Most notably, the PTG CVD score had a sensitivity of 82.5% and specificity of 96.8%, at a cutoff of 2, when used to detect CAD (P=0.0001; area under the receiver operating characteristic curve=0.967). The PTG spectral analysis markers were well-correlated to other autonomic nervous system and endothelial function markers. CAD diabetic patients (n=27) had a lower PTGi value compared with the CAD non-diabetic patients (n=38): and patients that underwent CABG (n=18) had a higher PTGi value compared with the CAD without CABG surgery patients (n=47).

Conclusion:

The spectral analysis of the photoplethysmography method is noninvasive, fast, operator-independent, and cost-effective, as only an oximeter and a galvanic skin response device are required in order to assess in a single testing the autonomic nervous system and endothelial function. The spectral analysis techniques used on the photoplethysmogram, as outlined in this study, could be useful when used alongside conventional known cardiovascular tests.

### REFERENCES

1. W C Chumlea, S S Guo, R J Kuczmarski, K M Flegal, C L Johnson, S B Heymsfield, H C Lukaski, K Friedl and V S Hubbard Body composition estimates from NHANES III bioelectrical impedance data. International Journal of Obesity (2002) 26, 1596-1609
2. Allen, J., Photoplethysmography and its application in clinical physiological measurement. Physiological Measurement, 2007. 28(3): p. R1-R39.
3. Foo, J. Y. and C. S. Lim, Dual-channel photoplethysmography to monitor local changes in vascular stiffness. J Clin Monit Comput, 2006. 20(3): p. 221-7.
4. Kubelka, P., New contributions to the optics of intensely light-scattering materials. J Opt Soc Am, 1948. 38(5): p. 448-57.
5. Cejnar, M., H. Kobler, and S. N. Hunyor, Quantitative photoplethysmography: Lambert-Beer law or inverse function incorporating light scatter. Journal of Biomedical Engineering, 1993. 15(2): p. 151-154.
6. Chan, G. S. H., et al., Automatic detection of left ventricular ejection time from a finger photoplethysmographic pulse oximetry waveform: comparison with Doppler aortic measurement. Physiological Measurement, 2007. 28(4): p. 439-452.
7. Wang, L., et al., Noninvasive cardiac output estimation using a novel photoplethysmogram index. Conf Proc IEEE Eng Med Biol Soc, 2009. 2009: p. 1746-9.
8. Elgendi, M., On the analysis of fingertip photoplethysmogram signals. Curr Cardiol Rev, 2012. 8(1): p. 14-25.
9. Takazawa, K., et al., Assessment of vasoactive agents and vascular aging by the second derivative of photoplethysmogram waveform. Hypertension, 1998. 32(2): p. 365-70.
10. Curt Diehm, F. Gerry R. Fowkes, William R. Hiatt, Björn Jönsson, Philippe Lacroix, Benoit, Victor Aboyans, Michael H. Criqui, Pierre Abraham, Matthew A. Allison, Mark A. Creager, Stoffers and Diane Treat-Jacobson. Measurement and Interpretation of the Ankle-Brachial Index. Circulation. 2012

1. A medical device system comprising at least two technologies wherein at least one technology is based on galvanic skin response measurement and at least one technology is based on oximeter wave form measurement wherein software cross analyzes the results to assess a level of homeostasis for an individual.

2. A system as claimed in claim 1 wherein a series of medical devices measure a variety of markers using different technologies and software compiles the results of these to provide a homeostasis score.

3. A system as claimed in claim 1 comprising at least a pulse oximeter to provide a vascular waveform in combination with other biosensors and software.

4. A system as claimed in claim 1 which galvanic skin response is assessed using a change in voltage polarity during measurement.

5. A system as claimed in claim 1 which the oximeter uses photoelectrical plethysmography in spectral analysis.

6. A system as claimed in claim 1 comprising at least 4 biosensors wherein signal processing-analysis is managed by software.

7. A system as claimed in claim 6 wherein the technologies include a) galvanic skin response, b) bioimpedance in tetrapolar mode c) oximeter sensor and d) blood pressure using oscillometric measurements.

8. Use of a system as claimed in claim 1 to establish a homeostasis score for an individual.

9. Use of homeostasis score markers to detect diabetes and cardiovascular diseases.

10. Use of homeostasis score markers to monitor treatment of diabetes and cardiovascular diseases.

11. Use of a medical device system as claimed in claim 1 wherein the software analyzes the results to calculate assess a patient and provides the results as a score using the homeostasis score markers to detect or monitor treatment of diabetes and cardiovascular diseases.
